(19) United States
(12) Patent Application Publication
(10) Pub. No.: US 2017/0042618 A1
(43) Pub. Date: Feb. 16, 2017

(54) DISPOSABLE SLEEVE FOR PROTECTING SURGICAL LASER DELIVERY FIBER ASSEMBLY, AND LASER DELIVERY FIBER WITH METAL TIP

(71) Applicant: Joe Denton Brown, Panama City Beach, FL (US)

(72) Inventor: Joe Denton Brown, Panama City Beach, FL (US)

(21) Appl. No.: 15/234,455

(22) Filed: Aug. 11, 2016

Related U.S. Application Data
(60) Provisional application No. 62/315,083, filed on Mar. 30, 2016.
(60) Provisional application No. 62/287,675, filed on Jan. 27, 2016.
(60) Provisional application No. 62/209,171, filed on Aug. 24, 2015.
(60) Provisional application No. 62/203,686, filed on Aug. 11, 2015.

(51) Int. Cl. A61B 18/22; A61B 90/00

(52) U.S. Cl. CPC A61B 18/22; A61B 90/04; A61B 2018/00547

(57) ABSTRACT

A disposable sleeve is fitted over a surgical optical fiber assembly before carrying out a treatment procedure. The optical fiber assembly includes an optical fiber, a laser-transparent protective cap that surrounds the fiber tip, and any additional components assembled to the optical fiber and protective cap, such as a coolant jacket. The disposable sleeve includes a transparent end portion corresponding to the transparent protective cap to permit passage of laser light, the sleeve protecting the surgical optical fiber and the protective cap in order to enable re-use of the surgical optical fiber. After the surgical procedure is performed, the sleeve is removed from the optical fiber assembly and the fiber is sterilized. Another sleeve is then placed over the optical fiber assembly so that the optical fiber assembly may be re-used to carry out a second treatment procedure. The disposable sleeve may optionally be preloaded with an index matching and/or cooling material to modulate the laser output, in which case a pressure-relief mechanism such as a spring-biased sealing gasket may be provided to accommodate expansion of the index matching and/or cooling material upon heating during a treatment procedure. The optical fiber assembly may include a side-firing fiber tip, and the fiber tip may optionally be reflectively coated or provided with a metal reflector.

This application claims the benefit of provisional U.S. Patent Appl. Ser. No. 62/315,083, filed Mar. 30, 2016, provisional U.S. Patent Appl. Ser. No. 62/287,675, filed Jan. 27, 2016, provisional U.S. Patent Appl. Ser. No. 62/209,171, filed Aug. 24, 2015, and provisional U.S. Patent Appl. Ser. No. 62/203,686, filed Aug. 11, 2015, each of which is incorporated herein by reference.

### BACKGROUND OF THE INVENTION

1. Field of the Invention

The invention relates to a disposable sleeve placed over an optical fiber assembly that includes, but is not limited to, an optical fiber to which a laser-transparent protective cap has been affixed. The laser-transparent protective cap at least partially encloses a tip of the fiber. The fiber tip may be an axial, radial or side-firing tip, optionally with assisted cooling. The fiber tip may be a conventional fiber tip or a fiber tip with a dielectric reflective coating or metal reflector. The optical fiber assembly, including the protective cap, may be of the type used in vascular, benign prostate hyperplasia (BPH), and other laser treatments.

The invention also relates to a laser treatment method utilizing optical fiber assembly that includes an optical fiber having a fiber tip enclosed within a laser-transparent protective cap, in which a disposable sleeve is placed over the optical fiber assembly before inserting the assembly into a patient for treatment, and in which the disposable sleeve is removable from the assembly after the treatment has been completed. The fiber tip may be a conventional axial, radial, or side-firing tip, with or without an additional dielectric or metal reflective layer.

The disposable sleeve and treatment method of the invention have the advantage of enabling reuse of optical fibers and optical fiber assemblies that would otherwise only be usable for a single treatment or surgical procedure.

Because the disposable sleeve is not welded to the fiber, it can be made of different transparent materials than those included in the fiber. For example, the disposable sleeve may be made of sapphire, which is able to withstand higher temperatures than fused silica.

The disposable sleeve also provides additional options for modulating the laser output, including rapid translation or rotation of the fiber or fiber assembly within the sleeve, and preloading of the sleeve with an index matching material.

2. Description of Related Art

In order to order lower the cost of surgical laser treatment procedures, there is an urgent need for a way to safely re-process the single-use optical fibers and fiber assemblies used in such treatment procedures. The price of an optical fiber or fiber assembly typically ranges from $100 to $1000 or higher. However, because the fibers are often damaged during treatment, and because the distal delivery tips with protective caps are often too complicated to remanufacture, it has not been possible to economically refurbish used surgical optical fibers and bring them back to original specifications. As a result, the used fibers and/or fiber assemblies must be permanently discarded and a new fiber or fiber assembly obtained for each procedure.

The term “fiber assembly” as used herein refers to the fiber itself, and any elements such as a fiber-tip protective cap or ferrule and/or a cooling sheath that are attached-to and insertable with the fiber during treatment, and that are not removed after treatment. Numerous such protective caps, ferrules, and/or cooling sheaths are known, with examples being illustrated in FIGS. 1-3.

FIG. 1 shows an example of the laser delivery fiber tip assembly 20 of a known radial firing optical fiber. The entire length of the fiber from the distal delivery end to the connector end (not shown) is typically three meters or more. The quartz tip of the fiber is shaped into a conical tip 16 that radially deflects laser energy propagating through the fiber and causes the laser energy to exit the fiber in a radial pattern, indicated by arrows 18, due to total internal reflection from the angled surface of the tip. An outer buffer layer of the fiber terminates in a laser-transparent protective cap 14 that permits passage of the laser energy while preventing fluid from contacting the fiber tip 16. Laser energy travels down the fiber core 10 and is transmitted from the conical tip 16 through the transparent protective cap 14.

Radial firing tips are a great choice for treating conditions such as varicose veins. The rounded tip of protective cap 14 allows the surgeon to pass the fiber to the distal treatment site without the risk of perforating the vein. Furthermore, the radial tip 16 lowers the fiber's output density compared to a flat tip. Lowering the output power density helps to prevent perforation of the vein wall during treatment. Unfortunately, through continued use, the quartz tip of the fiber erodes and the radial pattern 18 becomes diffuse, causing power density to drop, which renders the fiber useless, or worse erodes sufficiently that the cap 14 punctures and allows outside fluids to flow in and contact tip 16, completely destroying the radial pattern 18. As a result, due to the high cost of the radial tip fiber, surgeons will resort to using the less expensive flat tip fiber, despite the sharp edges and higher power density, because it can more easily be re-terminated and therefore re-used.

FIG. 2A shows an alternative tip in the form of a side firing fiber tip with a single facet 17 rather than a conical surface. The side-firing tip localizes the energy output of the fiber to one side as is required in certain types of surgical procedures or treatments, but otherwise has advantages and disadvantages similar to those of the radial tip fiber, including the disadvantages of relatively high cost, tip erosion, possible puncturing of the cap 14. Elements common to FIGS. 1 to 3 have been given the same reference numbers.

FIG. 2B shows a variation of the side firing fiber tip of FIG. 2A, in which the fiber tip is provided with a reflective coating 25. The addition of a reflective coating was proposed by Joe D. Brown and Stacy Childs in U.S. patent application Ser. No. 08/111,884, filed Aug. 26, 1993, and now abandoned. As disclosed in the 1993 patent application, the fiber 10 is enclosed within a transparent flexible or rigid capillary guide 14′ to form an optical fiber assembly, the capillary guide being either closed or arranged to include a cooling port through which cooling or irrigation fluid could be passed. Variations of the tip assembly disclosed by Brown and Childs in U.S. patent application Ser. No. 08/111,884, including multiple “capsules” placed over the beveled tip, are illustrated in U.S. Pat. Nos. 7,909,817 and 8,529,561.

FIG. 3A shows a variation of the fiber arrangement of FIG. 2A that further includes assisted cooling. In the arrangement shown in FIG. 3, a surgical fiber assembly with a side-firing tip corresponding to the one shown in FIG. 2A is further enclosed with a coolant jacket 19. Coolant 15, such as saline or water, travels coaxially relative to the fiber within the coolant jacket 19 and exits the coolant jacket 19 at the laser output. The coolant helps to slow the erosion of protective cap 14.

Laserscope sells a product called the “Moxy” that employs the arrangement of FIG. 3A in treating benign prostate hyperplasia (BPH). To treat BPH, surgeons are required to vaporize tissue with high power focused laser energy. However, while the cooling has the positive effect of reducing the wear on the laser-transparent protective cap 14, it also has the negative effect of cooling the target tissue 40 in the laser path 18, making it more difficult to achieve the intended vaporization.

Furthermore, as illustrated in FIGS. 3B and 3C, a problem arises that, during BPH surgery, the enlarged lobes of the prostate, known as “kissing lobes” as shown in FIG. 3A, can squeeze the “Moxy” instrument, i.e., coolant jacket 19 and the optical fiber and protective cap 14 enclosed therein, possibly causing sticking of tissue from the kissing prostate gland and interference with free movement of the optical fiber assembly with the coolant jacket 19. The problem of sticking tissue is a major cause of premature fiber failure.

As explained below, the present invention involves a disposable sleeve that fits over the optical fiber assembly, including at least a distal section of the optical fiber, the laser-transparent protective cap, and any coolant jacket, in order to reduce damage to the protective cap or fiber, including damage caused by erosion. The disposable sleeve is similar to a condom in that it is fitted over the optical fiber assembly before use, is inserted with the optical fiber assembly during use, and normally removed after use (although, unlike a condom, the disposable sleeve may in certain circumstances, such as when the fiber tip includes a damage-resistant metal reflector, be kept on for several procedures). Single-use protective sleeves have previously been used to protect instruments in surgical contexts, but not to protect laser fibers or laser fiber assemblies. The closest related art is perhaps a disposable sleeve sheath sold by Vision-Sciences and called the EndoSheath®, which is disposable but which fits over an endoscope and is not designed to be placed on an individual optical fiber or optical fiber assembly. The present invention has a similar protective effect, but is designed to fit over the fiber or fiber assembly rather than an endoscope or other fiber delivery tool.

In addition to the disposable endoscope sheath sold by Vision-Sciences, it has been proposed to provide sacrificial sheaths that glow or emit radiation when subjected to excessive heat in order to provide an early warning of overheating at the treatment site, as well as a variety of different permanently-affixed protective caps, sleeves, or ferrules that have a protective function similar to that of protective cap 14 illustrated in FIGS. 1, 2, and 3A. Sacrificial sheaths are disclosed, for example, in the inventor's U.S. Patent Publication Nos. 2011/0184310; 2011/0213349; and 2013/0218147. However, neither the conventional permanently-affixed protective caps nor the previously proposed sacrificial sheaths, enables re-use of the fibers. While the permanently affixed cap or tip might protect the fiber itself from excessive damage during use, the cap or tip is still exposed to damage, thereby preventing more than a single use of the entire assembly without expensive refurbishing. Sacrificial sheaths provide a useful way to monitor conditions during treatment, but also do not enable reuse of the fiber.

### SUMMARY OF THE INVENTION

It is accordingly an objective of the invention to provide a way to enable re-use of an optical fiber assembly, or an optical fiber, that, without application of the invention, would only be used once or a very limited number of times (hereinafter referred to as a single-use fiber).

This objective is achieved by providing a disposable sleeve that is arranged to fit over the optical fiber assembly or optical fiber so as to be inserted with the optical fiber assembly, and a method of using the disposable sleeve to enable multiple uses of single use fibers.

The terms “optical fiber assembly” (or “surgical fiber assembly”) as used herein shall refer to an optical fiber and to any components or elements affixed to the fiber that are not intended or required to be removed from the fiber after a surgical or treatment procedure, including laser-transparent protective caps and coolant sheaths or jackets. For example, the term “optical fiber assembly” encompasses not only the radial and side-firing optical fibers and protective caps of FIGS. 1 and 2B, but also the reflectively-coated fiber tip assembly of FIG. 2B, the fiber tip assembly with metal reflector illustrated in FIG. 2C, and the “Moxy” instrument of FIGS. 3A and 3C, in which the fiber and protective cap are situated within a coolant jacket.

In one preferred embodiment of the invention, a disposable sleeve is fitted over a laser-transparent protective cap at the distal or treatment end of the fiber, so as to prevent damage to the protective cap and the fiber tip enclosed within, and thereby enable re-use upon of the fiber/protective cap assembly upon re-sterilization.

In a further preferred embodiment of the invention, the fiber has a conical or angled tip and the disposable sleeve fits over the laser-transparent protective cap that encloses the conical or angled tip. However, it will be appreciated that the invention is not limited to radially-tipped or side-firing fibers, and that it may also be applied to fibers having ball-shaped or spherical tips, or any other tip configuration, including fiber tips with a reflective coating as illustrated in FIG. 2B and fiber tips with a metal reflector, as illustrated in FIG. 2C.

Those skilled in the art will appreciate that the use of a metal reflector at the fiber tip provides a number of advantages relative to a bare angled tip or dielectric reflective coating. Because the metal reflector is not subject to forward scatter, it offers the ability to better control or more freely vary the angle at which the laser is reflected out of the fiber, enabling use of relatively inexpensive fibers having plastic or silica cladding with a relative high numerical aperture (na). For example, a metal reflector can be arranged to provide an exit angle of greater than 90°, or multiple selected exit angles.

The disposable sleeve may be configured to modulate the laser output, in order to control the power density of radiation applied to a particular area of tissue. Alternatively, or in addition, the disposable sleeve may be pre-loaded with an indexing material, i.e., a material having a predetermined index of refraction for modulating the laser output, or a coolant with desired optical properties.

To provide pressure relief, the pre-loaded portion of the sleeve may be provided with a spring-biased gasket that permits the indexing material to expand upon heating.

In another preferred embodiment of the invention, the fiber has a side-firing tip and is included in a “Moxy” arrangement with assisted cooling, and the disposable sleeve fits over a coolant sheath that surrounds the fiber and protective cap. The addition of a disposable sleeve permits translation or rotation of the “Moxy” assembly within the sleeve, with the result that the target area may be expanded by rapid reciprocal movement of the fiber assembly within the disposable sleeve to increase power and vaporization rates without increasing power density.

In one implementation of the moving “Moxy” assembly, the movement may be a ratcheting movement achieved by adding a ratcheting mechanism to a Luer fitting coupling the sleeve and cap. The ratcheting mechanism rotates the sleeve and cap in one direction and just the cap is a second direction so as to constantly change the area of the sleeve exposed to the laser, which allows tissue to be burned-off for a self-cleaning effect.

In each of the preferred embodiments of the invention, the disposable sleeve includes a transparent section at the distal end in at least the area of the sleeve that is in the path of radiant energy as it passes through to the tissue being treated. The sleeve may be made of any material capable of protecting the optical fiber assembly during a single treatment, such as polyurethane or a similar material.

It will be appreciated by those skilled in the art that the term “disposable” refers not just to the fact that the material can be disposed of (any material, even diamonds, can be disposed of), but rather to the relative cost of the sleeve and the ease with which the sleeve can be removed from the optical fiber assembly. In particular, the cost of the sleeve should be a fraction of the cost of the optical fiber assembly, and the sleeve should be easily removable from the optical fiber assembly for disposal in a clinical setting, without damaging the optical fiber assembly.

Although the disposable or single-use sleeve of the invention will reduce contamination of the fiber, it is still possible for the fiber to accidentally pick up debris, which can become trapped between the fiber and a new disposable sheath. As a result, use of the disposable sheath does not necessarily eliminate the need to provide a means for detecting thermal runaway caused by the presence of the debris. Thus, the method of the invention may include a step of monitoring for overheating during a surgical procedure. By way of example and not limitation, the monitoring may include use of a blackbody radiation detector at the proximal end of the fiber or monitoring of visible radiation emitted by the fiber or sleeve upon overheating, which can be used to generate a laser interrupt signal or to provide a warning to the operator of the laser.

While a specific embodiment of the invention is described in connection with BPH surgery, it is intended that the invention not be limited to a particular type of laser surgery, or to applications involving particular treatment laser wavelengths. The fibers to which the invention may be applied typically are used with 400 nm to 2100 nm lasers, but the invention may also be applied to applications involving laser wavelengths of less than 400 nm and greater than 2100 nm.

### BRIEF DESCRIPTION OF THE DRAWINGS

FIG. 1 shows a conventional radial tip surgical optical fiber.

FIG. 2A shows a conventional side-firing surgical optical fiber.

FIG. 2B shows conventional side-firing surgical optical fiber assembly with a reflectively coated fiber tip.

FIG. 2C shows a side-firing surgical optical fiber assembly to which a metal reflector has been added.

FIG. 3A shows a side-firing laser tip with assisted cooling.

FIGS. 3B and 3C illustrate the problem with conventional BPH surgery using assisted cooling.

FIG. 4 shows the radial tip surgical optical fiber of FIG. 1, after it has been inserted into a disposable sleeve according to the principles of a preferred embodiment of the present invention.

FIG. 5 shows the side-firing laser tip with assisted cooling of FIG. 3, after it has been inserted into a variation of the disposable sleeve of FIG. 4.

FIG. 6 illustrates use of the arrangement of FIG. 5 for BPH surgery.

FIG. 7 is a cross-sectional side view of an arrangement in which a disposable sleeve is preloaded with an output-modulating index-matching and/or cooling material.

FIG. 8 is a cross-sectional side view of FIG. 7, after insertion of an optical fiber.

### DETAILED DESCRIPTION OF THE PREFERRED EMBODIMENTS

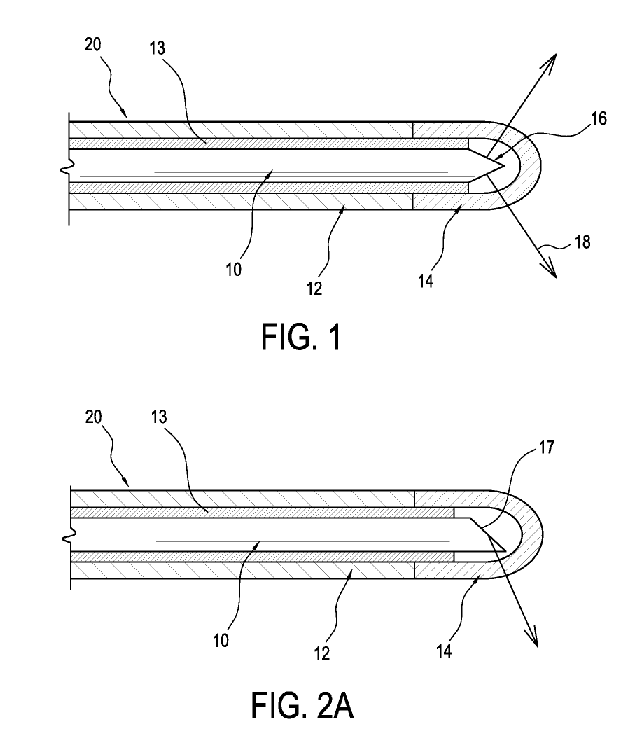

As illustrated in FIG. 4, a radial tip surgical optical fiber assembly 20 of the type illustrated in FIG. 1, including a fiber core 10 having a radial tip 16, a cladding layer 13, and a laser-transparent protective cap 14, is inserted into a disposable sleeve 34 that includes a laser-transparent distal end section 36. With the disposable sleeve 34 over the optical fiber assembly 20, the fiber cap 14 will remain pristine while the transparent tip of the sleeve 34 will now erode. If, at any time, too much erosion occurs to end section 36 of the disposable sleeve 34 occurs, then the sleeve can be readjusted to a new unused transparent tip 36 material, or the disposable sleeve can simply be replaced with another disposable sleeve 34.

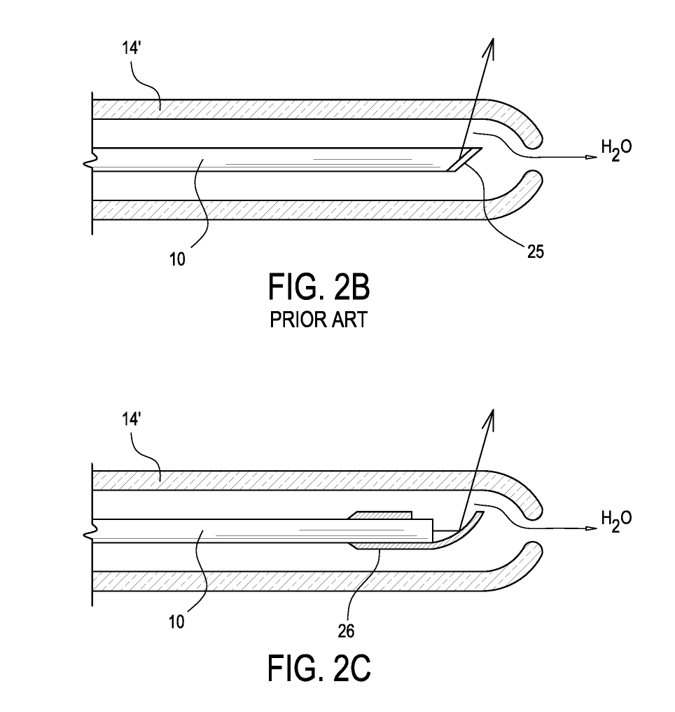

Instead of a bare fiber tip, it will be appreciated that a fiber tip with a reflective coating 25 of the type illustrated in FIG. 2B and described above, or a fiber tip with a metal reflector 26 as illustrated in FIG. 2C, may be included in the radial tip surgical optical fiber assembly 20 of FIG. 4. As illustrated in FIG. 2C, the metal reflector includes a sleeve portion that fits over the fiber 10 and an extension that is bent at a predetermined angle, or angles, to reflect light exiting the fiber at the predetermined angle(s). Suitable metals for the reflector 26 include but are not limited to gold, aluminum, and alloys thereof. Although an open-ended, transparent, quartz or sapphire cap 14′ that facilitates coolant flow is illustrated in FIG. 2C, the optical fiber assembly may also include a closed cap and the cap is not limited to a particular material or construction.

As shown in FIG. 4, the disposable sleeve 34 can extend from the laser-transparent section 36 back toward the proximal end of the fiber and terminate at any desired location. This has the added benefit of preventing the optical fiber assembly 20 from making patient contact and improves the ability to ensure good reprocess sterility and eliminate cross contamination. When the disposable sleeve 34 no longer has a sufficiently protective effect, or after completion of a surgical procedure or treatment, the sleeve may simply be removed from the optical fiber assembly 20 so that the optical fiber assembly can be sterilized for re-use.

If the procedure permits sterilization by disinfectant, then the fiber connector (not shown) that couples the optical fiber to the laser source machine could even be left permanently on the machine to help prevent damage to the connector or laser during sterilization or handling. This reduction in the possibility of damage to the fiber connector helps solve the problem that, if connector damage goes unnoticed, firing into the damaged connector could damage the laser or worse cause injury to the patient and/or operator.

Because the disposable sleeve 34 is not welded to the fiber, it can be made of different transparent materials than those included in the fiber. For example, the disposable sleeve may be made of sapphire, which is able to withstand higher temperatures than fused silica.

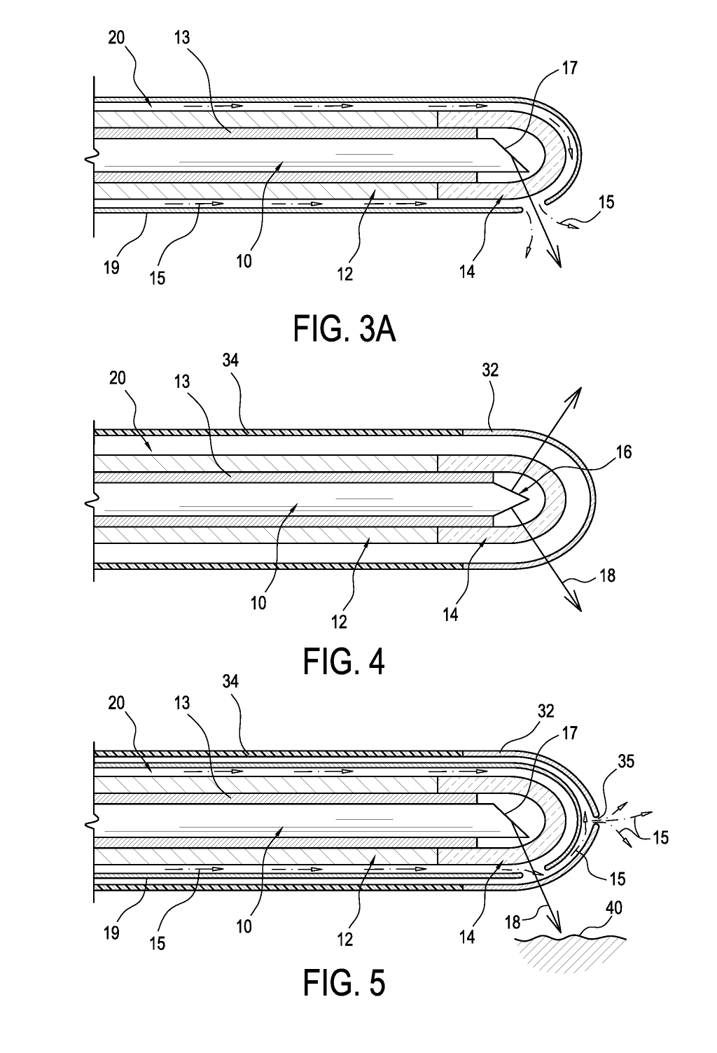

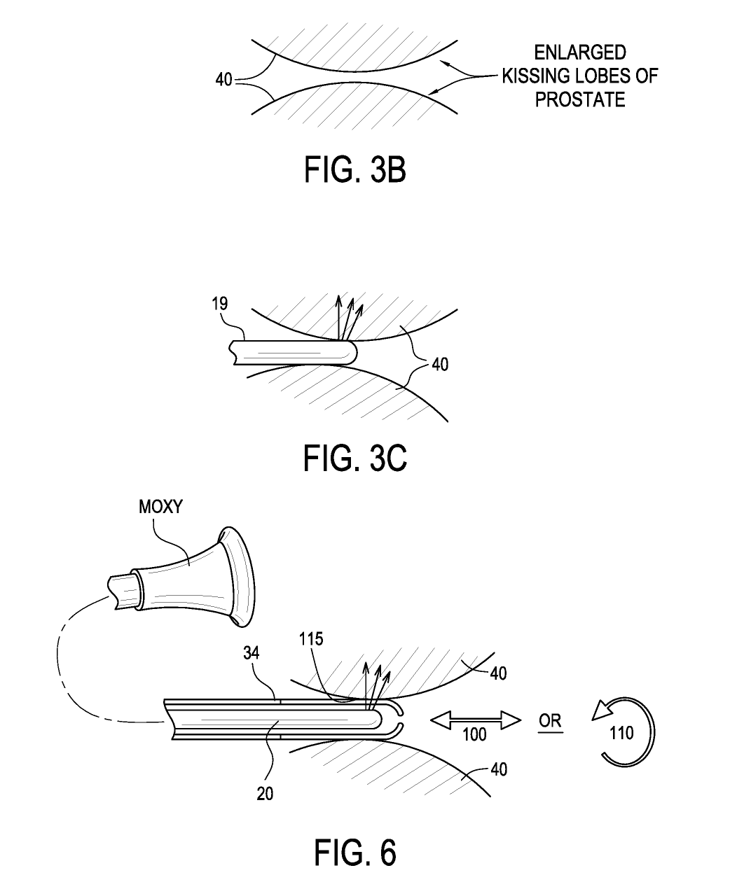

FIG. 5 shows an example of the application of the disposable sleeve to an optical fiber having a side-firing fiber tip 16 and a protective cap 14 with an assisted cooling jacket 16 of the type shown in FIG. 3, according to another preferred embodiment of the invention. The disposable sleeve 34 of this embodiment again has a transparent tip 32, but the transparent tip 32 is provided with a coolant exit hole 35. Since the sheath is disposable and the transparent tip can be adjusted toward or clean surface or simply exchanged for another sheath, the cooling can now be directed away from the target tissue 40, preventing the coolant from directly cooling the target tissue 40 and therefore providing improved vaporization. While the transparent tip 32 is now subject to faster erosion, this effect can easily be compensated for by disposing of and replacing the sleeve 34 or transparent tip 32 as often as necessary. The sleeve 34 also has the advantage of preventing cap 14 from being perforated during insertion, avoiding the problem that, if the cap 14 perforates, fluids will fill the cap and the cap will heat up and blow off the fiber 30.

As illustrated in FIG. 6, when the optical fiber assembly with assisted cooling is used as a “Moxy” instrument for BPH surgery, the addition of disposable sleeve 34 has the further advantage of allowing freer movement of the optical fiber assembly 20 relative to the “kissing lobes” 40 of the prostate gland, while preventing tissue from sticking to the optical fiber assembly 20 with the sleeve. This allows translation and/or rotation of the fiber or fiber assembly 20 within the sleeve, as indicated by respective arrows 100 and 110. As a result, the laser output can be modulated by rapid, reciprocal, sliding or rotating to effectively enlarge the spot size, which will help keep the surface of the clean and lower power density. In addition, movement of the fiber or fiber assembly permits a user to increase power while maintaining a given power density, resulting in faster vaporization rates.

The use of a disposable sleeve may have the further benefit of reducing the power density of the laser, by making the sleeve from a material having appropriate optical properties, or by preloading the sleeve with a material having optical properties, such as index of refraction, that result in modulation of the laser output. One advantage of reducing the power density of the laser in this manner is that the sleeve may advantageously be used with a flat tipped laser, to reduce the power density.

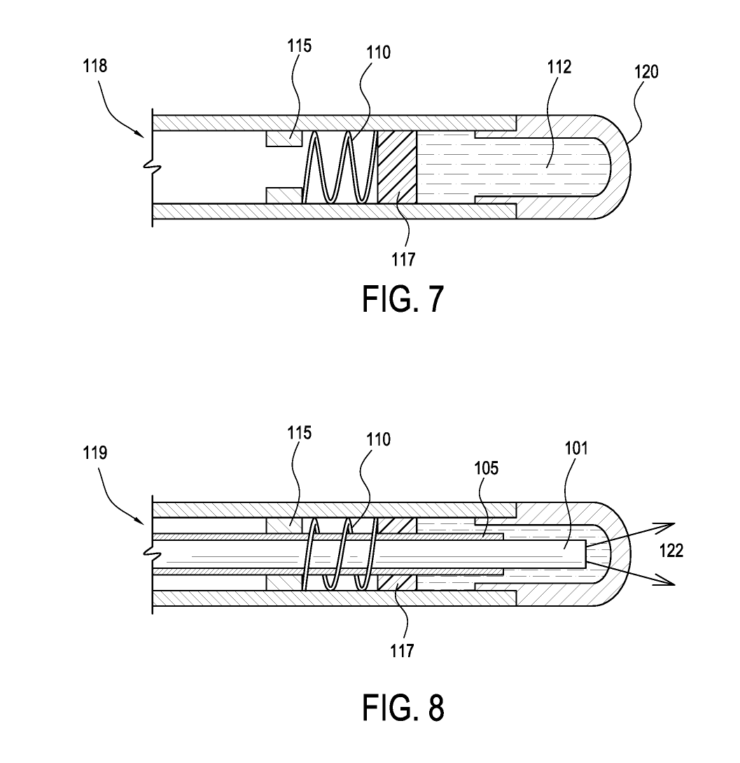

An example of a preloaded disposable sleeve 120 is illustrated in FIG. 7. A front section of the sleeve is sealed by a gasket 117 to form a compartment or chamber that is preloaded or filled with a material 112 having desired optical and/or coolant properties, such as index matching. Material 112 may be a liquid, gel, or any other fluid material having the desired properties. To accommodate expansion of the material 112 and provide pressure relief upon heating by the laser, the disposable sleeve preferably further includes an expansion spring 110 to accommodate axial displacement of the gasket 117. Spring 110 may be seated against a fixed gasket or annular structure 115. Those skilled in the art will appreciate that the spring-biased gasket may be replaced by other pressure-relief arrangements, such as a pressure-relief valve.

As shown in FIG. 8, a fiber including a fiber 112 including core 111 and buffer 115 is inserted through spring-mounting gasket 115, expansion spring 110, and sealing gasket 117 into the chamber containing index matching and/or cooling material 112. During a treatment procedure, the laser is fired through the material 112 and through the sleeve 120, with the material 112 modulating the laser so as to reduce the power density. While FIG. 8 shows an end-firing fiber tip, it will be appreciated that the disposable sleeve of this embodiment may also be used with side-firing fiber tips, as well as fiber tips having any other configuration.

The present invention also provides a method of using the disposable sleeve of FIGS. 4-8. In a preferred embodiment, the method involves the steps of inserting an optical fiber assembly into the disposable sleeve, inserting the optical fiber assembly with the disposable sleeve into a patient and performing a surgical procedure or treatment, and removing the disposable sleeve from the optical fiber assembly following the surgical procedure or treatment. The method may further include the step of rapidly translating or rotating the fiber or fiber assembly during the treatment to increase the area targeted by the laser.

In one preferred embodiment, the movement may be a ratcheting movement that can be achieved by adding a ratcheting mechanism to a Luer fitting coupling the sleeve and cap. The ratcheting mechanism rotates the sleeve and cap in one direction and just the cap is a second direction so as to constantly change the area of the sleeve exposed to the laser, which allows tissue to be burned-off for a self-cleaning effect.

Even though the protective “single-use” sleeve is sterile when initial placed over the fiber, and protects the fiber against contamination, it may still be possible for the fiber to become contaminated and for debris to become trapped between the fiber and the sleeve when a new sleeve is placed over the fiber. As a result, the method of the invention may optionally include monitoring steps to detect thermal runaway, including for example monitoring for radiation emitted by the fiber or sleeve during overheating, at which time a signal may be generated that interrupts the laser or warns the operator to take necessary action. Monitoring may be carried out from the proximal end of the fiber, or by a monitor or sensor included at the distal end of the fiber adjacent the treatment site.

What is claimed is:

1. A disposable sleeve for an optical fiber assembly including a surgical optical fiber and a laser-transparent protective cap at a distal end of the optical fiber, comprising a sleeve-shaped protective structure that removably fits over optical fiber assembly at least at a distal end of the optical fiber assembly, the sleeve-shaped protective structure including a transparent end portion corresponding to the laser-transparent protective cap to permit passage of laser light, wherein said disposable sleeve is arranged to be protect the surgical optical fiber and the protective cap in order to enable re-use of the surgical optical fiber.

2. A disposable sleeve as claimed in claim 1, wherein the surgical optical fiber has a conical tip.

3. A disposable sleeve as claimed in claim 1, wherein the surgical optical fiber is a side-firing surgical optical fiber having a single-facet tip.

4. A disposable sleeve as claimed in claim 1, wherein the surgical optical fiber has a reflectively coated tip.

5. A disposable sleeve as claimed in claim 1, further comprising a metal reflector positioned at the distal end of the fiber to direct laser light exiting the fiber at one or more predetermined angles.

6. A disposable sleeve as claimed in claim 1, wherein the surgical optical fiber is a side-firing surgical optical fiber with assisted cooling having a coolant jacket surrounding the fiber and through which coolant is supplied, said coolant jacket including an opening at a distal end of the jacket and said sleeve being arranged to fit over said coolant jacket, and wherein said sleeve includes a fluid opening for passage of said coolant.

7. A disposable sleeve as claimed in claim 6, wherein said fluid opening in said sleeve is oriented away from a side of the sleeve through which the laser is fired.

8. A disposable sleeve as claimed in claim 1, wherein the disposable sleeve is made of a transparent material different than a transparent material included in the fiber.

9. A disposable sleeve as claimed in claim 8, wherein the different transparent material is sapphire.

10. A disposable sleeve as claimed in claim 1, arranged to be preloaded with an index matching and/or cooling material.

11. A disposable sleeve as claimed in claim 10, further comprising a gasket for sealing a front chamber of the disposable sleeve to contain the index matching and/or cooling material, and an expansion spring to accommodate movement of the gasket and provide pressure-relief in response to heating of the index matching and/or cooling material, the gasket maintaining a seal upon insertion of the optical fiber into the front chamber.

12. A method enabling reuse of a surgical optical fiber assembly that includes an optical fiber and a protective cap that encloses a tip of the fiber and that is affixed to the fiber, comprising the steps of:
before using the fiber, inserting the optical fiber assembly into a disposable sleeve having a laser-transparent section, and positioning the disposable sleeve such that the laser-transparent section is aligned with a path of laser energy to be emitted from the tip of the optical fiber during a surgical procedure;
performing at least one surgical procedure; and
removing the sleeve after the at least one surgical procedure without disassembling the optical fiber assembly.

13. A method as claimed in claim 12, wherein the sleeve is removed after a single surgical procedure.

14. A method as claimed in claim 12, further comprising the steps of:
sterilizing the fiber,
inserting the fiber into another disposable sleeve, and
performing another surgical procedure using the fiber.

15. A method as claimed in claim 12, wherein the optical fiber assembly includes a side-firing optical fiber tip, a protective cap surrounding the tip, and a coolant jacket surrounding the optical fiber and protective cap, and the surgical procedure is a benign prostate hyperplasia treatment procedure.

16. A method as claimed in claim 12, further comprising the step of moving the optical fiber within the disposable sleeve during the surgical procedure to increase an area targeted by the laser.

17. A method as claimed in claim 16, wherein the movement is a ratcheting movement in which the sleeve and cap rotate together in one direction and only the cap rotates in the second direction so as to constantly change the area of the sleeve exposed to the laser and allow tissue to be burned-off for a self-cleaning effect.

18. A method as claimed in claim 12, further comprising the step of preloading the disposable sleeve with an index matching and/or cooling material before insertion of the optical fiber into the disposable sleeve.

19. A method as claimed in claim 12, further comprising the step of monitoring for thermal runaway resulting from debris accidentally trapped between the optical fiber and the disposable sleeve.

20. An optical fiber assembly including a surgical optical fiber and a laser-transparent protective cap at a distal end of the optical fiber, wherein a metal reflector is positioned at the distal end of the optical fiber to reflect laser light exiting the fiber at one or more predetermined angles.

21. An optical fiber assembly as claimed in claim 20, wherein the one or more predetermined angles include an angle of greater than 90°.

22. An optical fiber assembly as claimed in claim 20, wherein the protective cap includes a cap for enabling flow of coolant or irrigation fluid.
